(19) United States
(12) Patent Application Publication
(10) Pub. No.: US 2007/0190645 A1
(43) Pub. Date: Aug. 16, 2007

(54) Vascular cell culture patterning substrate

(75) Inventors: Hideyuki Miyake, Tokyo (JP); Hideshi Hattori, Tokyo (JP); Hironori Kobayashi, Tokyo (JP); Yusuke Uno, Tokyo (JP)

Correspondence Address: LADAS & PARRY LLP, 224 SOUTH MICHIGAN AVENUE, SUITE 1600, CHICAGO, IL, 60604, US

(73) Assignee: DAI NIPPON PRINTING CO., LTD., Tokyo-To (JP)

(21) Appl. No.: 10/592,167

(22) PCT Filed: Mar. 10, 2005
(86) PCT No.: PCT/JP05/04193

§ 371 Date: Jan. 3, 2007

(30) Foreign Application Priority Data
Mar. 10, 2004 (JP) 2004-067011

(51) Int. Cl. C12N 5/06; C12N 5/08; C12M 3/00

(52) U.S. Cl. 435/325; 435/366; 435/289.1

(57) ABSTRACT

A vascular cell culture patterning substrate, which can efficiently form a plurality of blood vessels on one substrate. The vascular cell culture patterning substrate includes: a base material; a vascular cell adhesion portion formed in at least two substantially parallel lines on the base material, and having adhesive properties to a vascular cell which forms a blood vessel; and a vascular cell adhesion-inhibiting portion formed in between two adjacent vascular cell adhesion portions on the base material, and inhibiting adhesion to the vascular cell. The vascular cell adhesion-inhibiting portion contains a vascular cell adhesion-inhibiting material having vascular cell adhesion-inhibiting properties of inhibiting adhesion to the vascular cell.

### TECHNICAL FIELD

The present invention relates to a vascular cell culture patterning substrate, which is used for culturing vascular cells to form blood vessels.

### BACKGROUND ART

At present, cell cultures of various animals and plants are performed, and also new cell culture methods are in development. The technologies of the cell culture are utilized, such as to elucidate the biochemical phenomena and natures of cells and to produce useful substances. Furthermore, with cultured cells, an attempt to investigate the physiological activity and toxicity of artificially synthesized medicals is under way.

Some cells, particularly a lot of animal cells have the adhesion dependency of adhering to some materials and growing thereon, and cannot survive for a long period under a flotation condition out of organisms. For culturing cells having such adhesion dependency, a carrier to which cells can adhere is necessary, and in general, a plastic culture dish with uniformly applied cell adhesive proteins such as collagen, fibronectin and the like is used. It is known that these cell adhesive proteins act on cultured cells, make the cells adhere easily, and exert an influence on the form of cells.

On the other hand, there is a technology reported of adhering cultured cells only onto a small part on a base material and arranging them. By such a technology, it is made possible to apply cultured cells to artificial organs, biosensors, bioreactors and the like. As the method for arranging cultured cells, there is a method adopted in which a base material having a surface that forms a pattern different in easiness of adhesion to cells is used, cells are cultured on the surface of this base material and allowed to adhere only onto surfaces processed so that cells adhere, and thereby the cells are arranged.

For example, in the patent document 1, an electric charge-retaining medium on which an electrostatic pattern is formed is applied to culture cells for the purpose of proliferating nerve cells in a form of circuit and the like.

Furthermore, the patent document 2 tries to arrange cultured cells on a surface on which a cell adhesion-inhibiting or cell adhesive photosensitive hydrophilic polymer has been patterned by a photolithography method.

Furthermore, the patent document 3 discloses a cell culture base material on which a substance such as collagen and the like affecting on the adhesion ratio and form of cells is patterned, and a method for manufacturing this base material by a photolithography method. By culturing cells on such a base material, a larger amount of cells can be adhered on a surface on which collagen or the like is patterned, to realize patterning of cells.

When vascular cells for forming blood vessels are cultured by employing such methods to form blood vessels, blood vessels will be formed by culturing vascular cells on vascular cell culture portions patterned in a line form. In this case, however, when a plurality of blood vessels is formed on one substrate, cell pseudopods will be extended between cells adhering to the adjacent vascular cell culture portions. Accordingly, there is a problem that when vascular tissues are regenerated by stimulating the vascular cells on the vascular cell culture portions, the formed vascular tissues will be adhered to one another via pseudopods contacting with adjacent vascular lines so as to form blood vessels in a form different from objective blood vessels, or blood vessels may be cut by stress upon such adhesion, thus failing to form objective blood vessels. To solve this problem, when forming only one blood vessel on one substrate, pseudopods will not be generated between vascular cells adhering to a line pattern, thereby causing no adhesion between blood vessels. However, there is a problem of low production efficiency.
Patent Document 1: Japanese Patent Application Laid-Open (JP-A) No. 2-245181
Patent Document 2: JP-A No. 3-7576
Patent Document 3: JP-A No. 5-176753

### DISCLOSURE OF INVENTION

### Problem to be Solved by the Invention

Accordingly, it has been desired to provide a vascular cell culture patterning substrate, which can efficiently form a plurality of blood vessels on one substrate.

### Means for Solving the Problem

The present invention provides a vascular cell culture patterning substrate comprising: a base material; a vascular cell adhesion portion formed in at least two substantially parallel lines on the base material, and having adhesive properties to a vascular cell which forms a blood vessel; and a vascular cell adhesion-inhibiting portion formed in between two adjacent vascular cell adhesion portions on the base material, and inhibiting adhesion to the vascular cell,

wherein the vascular cell adhesion-inhibiting portion contains a vascular cell adhesion-inhibiting material having vascular cell adhesion-inhibiting properties of inhibiting adhesion to the vascular cell.

In the present invention, since the vascular cell adhesion-inhibiting material is contained in the vascular cell adhesion-inhibiting portion formed in between the vascular cell adhesion portions, by setting the width of the vascular cell adhesion-inhibiting portion appropriately, bonding of the vascular cells on adjacent vascular cell adhesion portions to one another can be prevented so that the vascular cells will not be ruptured, thus enabling to culture the vascular cells in an objective shape.

In the above-mentioned invention, it is preferable that the width of the vascular cell adhesion-inhibiting portion is in the range of 200 μm to 600 μm. Thereby, when other cells are disseminated and cultured in between the blood vessels that are formed by using the vascular cell culture patterning substrate of the present invention, oxygen or the like can be sufficiently supplied to the disseminated cells via the blood vessel. Thus, the reproduced tissues or the like can be formed. Moreover, according to the range, plurality of blood vessels can be formed efficiently on one substrate.

Further, in the above-mentioned invention, the vascular cell culture patterning substrate may be such that: a photocatalyst-containing vascular cell adhesion layer is formed on the base material; the photocatalyst-containing vascular cell adhesion layer contains at least: a photocatalyst; and a vascular cell adhesive material which has adhesive properties to a vascular cell, and is to be decomposed or denatured by an action of a photocatalyst upon irradiation with energy; and in the vascular cell adhesion-inhibiting portion, the vascular cell adhesive material have been decomposed or denatured by an action of a photocatalyst upon irradiation with energy. Furthermore, the vascular cell culture patterning substrate may be such that: a photocatalyst-containing layer and a vascular cell adhesion layer are formed on the base material; the photocatalyst-containing layer contains at least a photocatalyst; the vascular cell adhesion layer contains a vascular cell adhesive material which has adhesive properties to a vascular cell, and is to be decomposed or denatured by an action of a photocatalyst upon irradiation with energy; and in the vascular cell adhesion-inhibiting portion, the vascular cell adhesive material have been decomposed or denatured by an action of a photocatalyst upon irradiation with energy. Still furthermore, the vascular cell culture patterning substrate may be such that: a vascular cell adhesion layer is formed on the base material; the vascular cell adhesion layer contains a vascular cell adhesive material which has adhesive properties to a vascular cell, and is to be decomposed or denatured by an action of a photocatalyst upon irradiation with energy; and in the vascular cell adhesion-inhibiting portion, the vascular cell adhesive material have been decomposed or denatured by an action of a photocatalyst upon irradiation with energy.

In either of the above-mentioned cases, the vascular cell adhesion portions and the vascular cell adhesion-inhibiting portions can be formed easily by an action of a photocatalyst upon irradiation with energy so that a vascular cell culture patterning substrate preferable in terms of the production efficiency, the cost, or the like can be provided. Moreover, at the time of adhering and culturing the vascular cells on the vascular cell adhesion portions, by irradiating the energy onto the vascular cell adhesion-inhibiting portion, the vascular cells or the like adhered on the vascular cell adhesion-inhibiting portions can be removed so that the vascular cells can be cultured in a highly precise pattern, and thus it is advantageous.

The present invention further provides a method for manufacturing a blood vessel, wherein the vascular cell is cultured using the above-mentioned vascular cell culture patterning substrate.

In the present invention, by using the above-mentioned cell culture patterning substrate, a high quality blood vessel can be formed because, during formation of the blood vessels, there will be no adhesion of adjacent blood vessels or no rupture of the blood vessels due to the adhesion.

According to the present invention, since there will be no adhesion of adjacent blood vessels or no rupture of the blood vessels due to the adhesion, effects that the vascular cells can be cultured in an objective form because the following can be prevented: adhesion of the cell pseudopods, generated from the vascular cells adhered on the adjacent vascular cell adhesion portions, to one another; and contacts of the vascular cells adhered on the vascular cell adhesion-inhibiting portions and the vascular cells adhered on the vascular cell adhesion portions, due to adhesion of the vascular cells on to the vascular cell adhesion-inhibiting portions.

### BRIEF DESCRIPTION OF THE DRAWINGS

FIG. 1 is a plan view showing an example of the vascular cell culture patterning substrate of the present invention.

FIG. 2 is an explanatory diagram showing an example of a method for forming a vascular cell adhesion portion and a vascular cell adhesion-inhibiting portion of a vascular cell culture patterning substrate of the present invention.

FIG. 3 is a schematic sectional view showing an example of the photocatalyst-containing layer side substrate used in the present invention.

FIG. 4 is a schematic sectional view showing another example of the photocatalyst-containing layer side substrate used in the present invention.

FIG. 5 is a schematic sectional view showing another example of the photocatalyst-containing layer side substrate used in the present invention.

FIG. 6 is an illustration showing another example of the method for forming the vascular cell adhesion portion and vascular cell adhesion-inhibiting portion in the vascular cell culture patterning substrate of the present invention.

DESCRIPTION OF SYMBOLS

1: Base material
2: Vascular cell adhesion portion
3: Vascular cell adhesion-inhibiting portion

### BEST MODE FOR CARRYING OUT THE INVENTION

The present invention relates to a vascular cell culture patterning substrate, which is used in culturing vascular cells to form blood vessels, as well as a method for manufacturing blood vessels by using the vascular cell culture patterning substrate. Hereinafter, these will be explained respectively.

A. Vascular Cell Culture Patterning Substrate

First, a vascular cell culture patterning substrate of the present invention will be explained. The vascular cell culture patterning substrate of the invention comprises: a base material; a vascular cell adhesion portion formed in at least two substantially parallel lines on the base material, and having adhesive properties to a vascular cell which forms a blood vessel; and a vascular cell adhesion-inhibiting portion formed in between two adjacent vascular cell adhesion portions on the base material, and inhibiting adhesion to the vascular cell,

wherein the vascular cell adhesion-inhibiting portion contains a vascular cell adhesion-inhibiting material having vascular cell adhesion-inhibiting properties of inhibiting adhesion to the vascular cell.

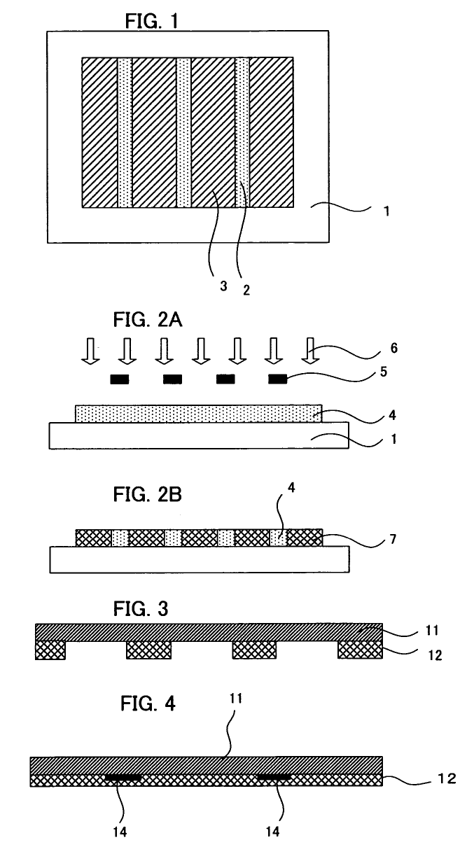

For example, as shown in FIG. 1, the vascular cell culture patterning substrate of the present invention comprises: a base material 1; at least two vascular cell adhesion portions 2, having adhesive properties to a vascular cell, formed on the base material 1 as substantially parallel lines; and vascular cell adhesion-inhibiting portions 3, which inhibits adhesion to the cells, formed in between the vascular cell adhesion portions 2, wherein the vascular cell adhesion-inhibiting portion 3 contains a vascular cell adhesion-inhibiting material having vascular cell adhesion-inhibiting properties.

At the time of adhering and culturing the vascular cells on the vascular cell adhesion portion of the vascular cell culture patterning substrate of the present invention, since the vascular cell adhesion-inhibiting material is contained in the vascular cell adhesion-inhibiting portion, the vascular cells can hardly be adhered on the vascular cell adhesion-inhibiting portion. Thus, for example, adhesion or the like of the vascular cells adhered on the vascular cell adhesion-inhibiting portion and the vascular cells adhered on the vascular cell adhesion portion can be prevented. Moreover, since adhesion of the cell pseudopods generated from the vascular cells adhered on the adjacent cell adhesion portions can also be prevented, adhesion among the adjacent blood vessels, rupture of the formed blood vessels due to the stress applied among the adjacent blood vessels by the adhesion or the like can be prevented. Therefore, plurality of blood vessels can be formed efficiently on one substrate.

In the present invention, it is preferable that the distance between the vascular cell adhesion portions, that is, the width of the vascular cell adhesion-inhibiting portion is set in the range of 200 μm to 600 μm, in particular, 300 μm to 500 μm. According to the width, at the time of culturing the vascular cells using the vascular cell culture patterning substrate of the present invention, the width of the formed blood vessels can be relatively narrow. Therefore, at the time of forming the tissues by disseminating other cells between these blood vessels, oxygen or the like can be supplied sufficiently to the disseminated cells by the blood vessels so that the cells in between the blood vessels can be cultured without necrosis or the like. Moreover, according to the gap in the range, there is an advantage that a large number of blood vessels can be formed efficiently on one substrate.

Hereinafter, each configuration of the vascular cell culture patterning substrate of the present invention will be explained in detail.

(Vascular Cell Adhesion-inhibiting Portion)

First, the vascular cell adhesion-inhibiting portion in the present invention will be explained. The vascular cell adhesion-inhibiting portion in the present invention is: a region having the vascular cell adhesion-inhibiting properties of inhibiting adhesion to a vascular cell; formed in between two adjacent vascular cell adhesion portions on the below-described base material; and containing the vascular cell adhesion-inhibiting material.

The vascular cell adhesion-inhibiting material has the vascular cell adhesion-inhibiting properties of inhibiting adhesion to a vascular cell. As the vascular cell adhesion-inhibiting material having the vascular cell adhesion-inhibiting properties, for example, a material having high hydration ability can be used. When the material having high hydration ability is used as the vascular cell adhesion-inhibiting material, a hydration layer, wherein water molecules gather around the vascular cell adhesion-inhibiting material, is formed. Usually, such a material having high hydration ability has higher affinity for water molecules than for vascular cells. Thus, the vascular cells cannot be adhered to the material having high hydration ability so that the adhesive properties to a vascular cell will be low. The hydration ability refers to a property of hydrating with water molecules, and the high hydration ability is intended to mean that the material is easily hydrated with water molecules.

As the vascular cell adhesion-inhibiting material, a material having water repellency, oil repellency or superhydrophilicity can also be used. This is because, by the water repellency, oil repellency or superhydrophilicity of the vascular cell adhesion-inhibiting material, the interaction between vascular cells and the vascular cell adhesion-inhibiting material can be made lower, thus decreasing adhesive properties to a vascular cell.

Here, it is preferable that the vascular cell adhesion-inhibiting material is contained in the vascular cell adhesion-inhibiting portion by about 0.01% by weight to 95% by weight, more preferably by 1% by weight to 10% by weight. Thereby, adhesion of the vascular cells between the vascular cell adhesion-inhibiting portions, contact or the like of the cell pseudopods generated from the vascular cells adhered on the adjacent vascular cell adhesion portions can be prevented.

The material having high hydration ability which can be used as the vascular cell adhesion-inhibiting material includes, for example, polyethylene glycol, amphoteric ionic materials having a betaine structure, phospholipid-containing materials, etc.

As the material having water repellency or oil repellency, for example, a material having a water-repellent or oil-repellant organic substituent can be used. Specific examples thereof include organopolysiloxanes exhibiting large strength obtained by hydrolyzing or polycondensating chloro- or alkoxysilanes by sol-gel reaction etc., as well as organopolysiloxanes obtained by crosslinking reactive silicone. As to the water repellency and the oil repellency of the material having the vascular cell adhesion-inhibiting properties, it is preferable that the contact angle with water is in general 80° or more, and in particular in the range of 100° to 130°. By having such contact angle with water, adhesion to a vascular cell can be inhibited.

Moreover, as the material having the superhydrophilic properties, organopolysiloxane, etc., whose organic substituent is decomposed by an action of a photocatalyst upon irradiation with energy or the like, can be presented. The superhydrophilic properties, which exhibit the vascular cell adhesion-inhibiting properties, preferably mean the contact angle with water of 10° or less. By having such contact angle with water, adhesion to a vascular cell can be inhibited.

In the case the organopolysiloxane, etc. have a contact angle with water of 15° to 120°, particularly in the range of 20° to 100°, it can be used as one having the vascular cell adhesive properties to a vascular cell. Therefore, by utilizing the change of the adhesive properties to a vascular cell due to the change of the contact angle with water of the organopolysiloxane, etc., the vascular cell adhesion-inhibiting portion and the vascular cell adhesion portion can be formed as it will be described later.

The contact angle with water referred to herein is a result obtained by using a contact angle measuring device (CA-Z model, manufactured by Kyowa Interface Science Co., Ltd.) to measure the contact angle of the material with water or a liquid having a contact angle equivalent to that of water (after 30 seconds from the time when droplets of the liquid are dropped down from its micro syringe), or a value obtained from a graph prepared from the result.

Here, examples of the method for forming the vascular cell adhesion-inhibiting portion are as follows: a method in which the vascular cell adhesion-inhibiting layer, containing the vascular cell adhesion-inhibiting material, is printed by a common printing method or the like; and a method in which the layer is formed in a pattern by, for example, the photolithography method. Moreover, for example, in the case the below-mentioned base material contains the vascular cell adhesion-inhibiting material, the base material may be used as the vascular cell adhesion portion. Moreover, the vascular cell adhesion-inhibiting portion may be formed by utilizing an action of a photocatalyst upon irradiation with energy. This will be explained later in detail.

(Vascular Cell Adhesion Portion)

Next, the vascular cell adhesion portion of the vascular cell culture patterning substrate of the present invention is described. The vascular cell adhesion portion in the present invention is a region formed on a base material described later and having adhesive properties to a vascular cell for forming a blood vessel. In the present invention, at least two vascular cell adhesion portions are formed as substantially parallel lines on the vascular cell culture patterning substrate. As used herein, the term “substantially parallel” refers not only to completely parallel but also to substantially parallel, that is, the two lines are not crossed in a region. It includes, for example, lines such as zigzag lines that exist without crossing one another. The term “substantially parallel” also refers to portions that are not crossed in a crossed structure such as a net-like structure.

The shape of the vascular cell adhesion portion is not particularly limited insofar as it is formed in a line form. The shape is selected suitably depending on the shape of an objective blood vessel. Usually, the line width of the vascular cell adhesion portion shall be about 10 μm to 5000 μm, especially 20 μm to 100 μm, particularly 40 μm to 60 μm. A line width of less than 10 μm is not preferable because adhesion of vascular cells is made difficult. A line width of greater than 5000 μm, on the other hand, is not preferable either because almost all vascular cells will be adheres to the vascular cell adhesion portion in a spread state, thus making the cultured vascular cells hardly formable in the form of a blood vessel.

In the present invention, in order to produce a preferable blood vessel, it is particularly preferable that the vascular cell adhesion auxiliary portion is provided in the vascular cell adhesion portion. The vascular cell adhesion auxiliary portion denotes a region having no adhesive properties to a vascular cell, formed as a minute pattern in the vascular cell adhesion portion. The vascular cell adhesion auxiliary portion is formed in a minute pattern to a degree not to inhibit the bonding between the vascular cells in the vascular cell adhesion portion at the time of adhering the vascular cells on the vascular cell adhesion portion. That is, to the degree that the vascular cells can be bound with each other also on the vascular cell adhesion auxiliary portion.

Generally, when vascular cells are adhered to the vascular cell adhesion portion and cultured to form a tissue, the vascular cells are gradually arranged from the outside toward inside of the vascular cell adhesion portion. For forming a tissue, individual vascular cells should be changed morphologically and arranged, and this morphological change of the vascular cell also gradually occurs from the edge part toward center part of the vascular cell adhesion portion. Accordingly, when the width of the vascular cell adhesion portion is large, a tissue may not be formed in the center part of the vascular cell adhesion portion because of insufficient arrangement of the vascular cells, or the vascular cells may fail to adhere to the center part of the vascular cell adhesion portion. Moreover, the morphological change of the vascular cells in the center part of the vascular cell adhesion portion may be insufficient. Therefore, by forming the vascular cell adhesion auxiliary portion described above, the vascular cells can be arranged or morphologically changed from the edge part of the vascular cell adhesion auxiliary portion. Thereby, the vascular cells can be cultured without generating defects or inferior morphological change. Moreover, the vascular cell adhesion auxiliary portion is formed such that vascular cells adjacent to one another via the vascular cell adhesion auxiliary portion are not prevented from being adhered to one another. Thus, the width of the finally cultured vascular cells can be the same as the width of the vascular cell adhesion portion.

The vascular cell adhesion auxiliary portion is formed preferably in a line form in the vascular cell adhesion portion. The shape of the line is not particularly limited and can be in the form of, for example, a straight line, a curved line, a dotted line, a broken line, etc. The line width of the vascular cell adhesion auxiliary portion is preferably in the range of 0.5 μm to 10 μm, more preferably 1 μm to 5 μm. The width larger than the above range is not preferable because the vascular cells adjacent to one another via the vascular cell adhesion auxiliary portion will hardly interact with one another on the vascular cell adhesion auxiliary portion. When the width is smaller than the above range, on the other hand, the vascular cell adhesion auxiliary portion will be hardly formed by pattern forming techniques described later.

The vascular cell adhesion auxiliary portion may be formed to have a convexoconcave pattern (for example, zigzag etc.) in plane. The term “in plane” refers to the surface of a base material or a surface analogous thereto. The average distance from the edge part of the concave portion to the edge part of the convex portion, of the convexoconcave pattern, may be such a distance that when vascular cells are adhered to the vascular cell adhesion portion, the vascular cells are aligned in the same direction as the line direction of the vascular cell adhesion portion, and the average distance is particularly preferably in the range of 0.5 μm to 30 μm. The average distance from the edge part of the concave portion to the edge part of the convex portion of the convexoconcave pattern is determined by calculating the average of measured distances from the lowermost bottom to the uppermost top of each concavoconvex, within the range of 200 μm of the edge portion of the vascular cell adhesion auxiliary portion.

Concerning the phrase “vascular cell adhesion portion having adhesive properties to a vascular cell”, the adhesive properties to a vascular cell may be imparted by, for example, biochemical properties. Moreover, the adhesive properties to a vascular cell may be imparted by physicochemical properties.

Such vascular cell adhesion portion, for example, may be obtained by forming a vascular cell adhesion layer containing a vascular cell adhesive material having adhesive properties to a vascular cell. Moreover, for example, when a base material described later has adhesive properties to a vascular cell, the surface of the base material can be used as the vascular cell adhesion portion. The method for forming the vascular cell adhesion layer includes general printing methods, photolithographic techniques, and patterning methods using the action of a photocatalyst upon irradiation with energy.

A material having adhesive properties to a vascular cell which can be also used as a base material described later includes various kinds of glass, plasma-treated polystyrene, polypropylene etc. As the vascular cell adhesive material used in the vascular cell adhesion layer, cell adhesive materials used in general cell culture substrates etc. can be used. Examples of materials adhering to vascular cells for example by physicochemical properties include hydrophilic polystyrene, poly(N-isopropylacrylamide), basic polymers such as polylysine, basic compounds such as aminopropyltriethoxysilane, N-(2-aminoethyl)-3-aminopropyltrimethoxysilane etc., and condensates containing thereof. The vascular cell adhesive material having adhesive properties to a vascular cell biochemically includes fibronectin, laminine, tenascin, vitronectin, an RGD (arginine-glycine-aspartic acid) sequence-containing peptide, a YIGSR (tyrosine-isoleucine-glycine-serine-arginine) sequence-containing peptide, collagen, atelocollagen, gelatin, and mixtures thereof, for example matrigel etc.

(Base Material)

Now, the base material used in the present invention is described. The base material used in the present invention is not particularly limited and may have vascular cell adhesive properties or vascular cell adhesion-inhibiting properties.

As such base material, for example as well as the above-described materials, an inorganic material such as metal and silicon, or an organic material typified by plastics and the like can be used.

Moreover, plasticity, transparency, etc. of the base material is appropriately selected according to the types and uses of the cell culture patterning substrate.

(Vascular Cell Culture Patterning Substrate)

The vascular cell culture patterning substrate of the present invention is not particularly limited in so far as it comprises a base material, the above-described vascular cell adhesion portion and the vascular cell adhesion-inhibiting portion, and if necessary, other suitable members etc. may be formed.

Here, in the present invention, the vascular cell adhesion portion and the vascular cell adhesion-inhibiting portion may be formed by decomposing or denaturing the vascular cell adhesion-inhibiting material by irradiating the energy, in a pattern of the vascular cell adhesion portion to be formed, to the vascular cell adhesion-inhibiting layer containing the vascular cell adhesion-inhibiting material, which inhibits the adhesion to a vascular cell, to be decomposed or denatured by an action of a photocatalyst upon irradiation with energy.

Moreover, in the present invention, the vascular cell adhesion-inhibiting portion may be formed by decomposing or denaturing the vascular cell adhesive material by irradiating the energy, in a pattern of the vascular cell adhesion-inhibiting portion to be formed, to the vascular cell adhesion layer containing the vascular cell adhesive material having the adhesive properties to a vascular cell, to be decomposed or denatured by an action of a photocatalyst upon irradiation with energy.

According to these methods, the vascular cell adhesion portion and the vascular cell adhesion-inhibiting portion can be formed easily so that a vascular cell culture pattering substrate preferable also in terms of the manufacturing efficiency, the cost or the like can be provided.

In the present invention, the latter method is particularly preferable. Thereby, at the time of manufacturing a blood vessel by adhering the vascular cells on the vascular cell adhesion portion of the vascular cell culture patterning substrate of the present invention, the adhesive properties to a vascular cell of the vascular cell adhesion-inhibiting portion can be lowered by affecting an action of a photocatalyst upon irradiation with energy onto the vascular cell adhesion-inhibiting portion, or the adhered vascular cells can be removed by an action of a photocatalyst.

A method for forming the vascular cell adhesion portion and the vascular cell adhesion-inhibiting portion by using the vascular cell adhesion layer containing the vascular cell adhesive material having the adhesive properties to a vascular cell, to be decomposed or denatured by an action of a photocatalyst upon irradiation with energy will be explained hereafter. As such embodiment, the following three embodiments can be presented. Each embodiment will be explained in detail.

### (1) FIRST EMBODIMENT

First, the first embodiment is the vascular cell culture patterning substrate, wherein: a photocatalyst-containing vascular cell adhesion layer is formed on the base material; the photocatalyst-containing vascular cell adhesion layer contains at least: a photocatalyst; and a vascular cell adhesive material which has adhesive properties to a vascular cell, and is to be decomposed or denatured by an action of a photocatalyst upon irradiation with energy; and in the vascular cell adhesion-inhibiting portion, the vascular cell adhesive material have been decomposed or denatured by an action of a photocatalyst upon irradiation with energy.

In this embodiment, since the photocatalyst-containing vascular cell adhesion layer contains the photocatalyst and the vascular cell adhesive material, a vascular cell adhesion-inhibiting portion having the vascular cell adhesion-inhibiting properties can be provided by decomposing or denaturing the vascular cell adhesive material by an action of the photocatalyst by irradiating the energy to the photocatalyst-containing vascular cell adhesion layer on the region to form the vascular cell adhesion-inhibiting portion. On the other hand, since the vascular cell adhesive material remains in the region without the energy irradiation, a vascular cell adhesion portion having preferable adhesive properties to a vascular cell can be provided. Therefore, without the need of a special device, a complicated process or the like, by irradiating the energy in a pattern, the vascular cell adhesion portion and the vascular cell adhesion-inhibiting portion can be formed easily.

Hereinafter, the photocatalyst-containing vascular cell adhesion layer and the base material used in this embodiment will be explained. Furthermore, the method for forming the vascular cell adhesion-inhibiting portion will be explained.

a. Photocatalyst-containing Vascular Cell Adhesion Layer

First, the photocatalyst-containing vascular cell adhesion layer used in this embodiment will be explained. The photocatalyst-containing vascular cell adhesion layer used in this embodiment contains at least a photocatalyst and the vascular cell adhesive material. Also, this layer is a layer which will be a vascular cell adhesion-inhibiting layer of having no adhesive properties to a vascular cell, that is, a layer inhibiting adhesion to a vascular cell, by decomposing or denaturing the vascular cell adhesive material by an action of a photocatalyst upon irradiation with energy.

The photocatalyst-containing vascular cell adhesion layer can be formed by coating, etc. a photocatalyst-containing vascular cell adhesion layer forming coating solution, containing a photocatalyst and a vascular cell adhesive material to be decomposed or denatured by an action of a photocatalyst upon irradiation with energy, onto a base material. The photocatalyst-containing vascular cell adhesion layer forming coating solution can be coated by a common coating method. For example, the spin coating method, the spray coating method, the dip coating method, the roll coating method, the bead coating method or the like can be used.

At the time, the film thickness of the photocatalyst-containing vascular cell adhesion layer can be selected optionally according to the kind, etc. of the vascular cell culture patterning substrate. It is in general about 0.01 μm to 1.0 μm, in particular, about 0.1 μm to 0.3 μm.

Hereinafter, each material used for the photocatalyst-containing vascular cell adhesion layer used in this embodiment will be explained.

(i) Vascular Cell Adhesive Material

First, the vascular cell adhesive material contained in the photocatalyst-containing vascular cell adhesion layer of this embodiment will be explained. The kind, etc. of the vascular cell adhesive material contained in the photocatalyst-containing vascular cell adhesion layer is not particularly limited as long as it has the adhesive properties to a vascular cell and it is decomposed or denatured by an action of a photocatalyst upon irradiation with energy so as to be the vascular cell adhesion-inhibiting material.

Here, “having adhesive properties to a vascular cell” means being good in adhesion to a vascular cell. For instance, when the adhesive properties to a vascular cell differ depending on the kind of vascular cells, it means to be good in the adhesion with target vascular cells.

The vascular cell adhesive material used in the present embodiment has such adhesive properties to a vascular cell. Those changed into vascular cell adhesion-inhibiting material having the vascular cell adhesion-inhibiting properties of inhibiting adhesion to vascular cells, by being decomposed or denatured by an action of a photocatalyst upon irradiation with energy, are used.

As such materials having the adhesive properties to a vascular cell, there are two kinds. One being material having the adhesive properties to a vascular cell owing to physicochemical characteristics and the other being material having the adhesive properties to a vascular cell owing to biochemical characteristics.

As physicochemical factors that determine the adhesive properties to a vascular cell of the materials having the adhesive properties to a vascular cell owing to the physicochemical characteristics, the surface free energy, the electrostatic interaction and the like can be cited. For instance, when the adhesive properties to a vascular cell is determined by the surface free energy of the material, if the material has the surface free energy in a predetermined range, the adhesive properties between the vascular cells and the material becomes good. If it deviates from the predetermined range, the adhesive properties between the vascular cells and the material will be lower, and the material will have vascular cell adhesion-inhibiting properties. As such changes of the adhesive properties to a vascular cell due to the surface free energy, experimental results shown in Data, for instance, CMC Publishing Co., Ltd. “Biomaterial no Saisentan”, Yoshito Ikada (editor), p. 109, lower part are known. As materials having the adhesive properties to a vascular cell owing to such a factor, for instance, hydrophilic polystyrene, poly (N-isopropyl acrylamide) and the like can be cited. When such a material is used, by the action of a photocatalyst upon irradiation with energy, for instance, a functional group on a surface of the material is substituted, decomposed or the like to cause a change in the surface free energy, resulting in one having the vascular cell adhesion-inhibiting properties.

When the adhesive properties between vascular cells and a material is determined owing to the electrostatic interaction or the like, for instance, the adhesive properties to a vascular cell are determined by an amount of positive electric charges and the like that the material has. As materials having the adhesive properties to a vascular cell owing to such electrostatic interaction, basic polymers such as polylysine; basic compounds such as aminopropyltriethoxysilane, N-(2-aminoethyl)-3-aminopropyltrimethoxysilane; and condensates and the like including these can be cited. When such materials are used, by the action of a photocatalyst upon irradiation with energy, the above-mentioned materials are decomposed or denatured. Thereby, for instance, an amount of positive electric charges present on a surface can be altered, resulting in one having the vascular cell adhesion-inhibiting properties.

As materials having the adhesive properties to a vascular cell owing to the biological characteristics, ones that are good in the adhesive properties with particular vascular cells or ones that are good in the adhesive properties with many vascular cells can be cited. Specifically, fibronectin, laminin, tenascin, vitronectin, RGD (arginine-glycine-asparagine acid) sequence containing peptide, YIGSR (tyrosine-isoleucine-glycine-serine-arginine) sequence containing peptide, collagen, atelocollagen, gelatin and mixture thereof, such as matrigel and the like, can be cited. When such material is used, by the action of a photocatalyst upon irradiation with energy, for instance, a structure of the material is partially destroyed, or a principal chain is destroyed or the like, resulting in one having the vascular cell adhesion-inhibiting properties.

Such a vascular cell adhesive material, though it differs depending on the kind of the materials and the like, is comprised in the photocatalyst-containing vascular cell adhesion layer normally in the range of 0.01% by weight to 95% by weight, and preferably in the range of 1% by weight to 10% by weight. Thereby, a region that contains the vascular cell adhesive material can be made a region good in the adhesive properties to a vascular cell.

(ii) Photocatalyst

Next, the photocatalyst contained in the photocatalyst-containing vascular cell adhesion layer of this embodiment will be explained. The photocatalyst used in this embodiment is not particularly limited as long as it can decompose or denature the above-mentioned vascular cell adhesive material by an action of a photocatalyst upon irradiation with energy.

Here, although the function mechanism of the photocatalyst represented by a titanium oxide to be described later is not always clear, it is considered that the carrier produced by the light irradiation changes the chemical structure of an organic substance by the direct reaction with a compound in the vicinity or by the active oxygen species generated in the presence of oxygen and water. In this embodiment, the carrier is considered to act on the vascular cell adhesive material.

As the photocatalyst that can be used in the present embodiment, specifically, for instance, titanium dioxide (TiO₂), zinc oxide (ZnO), tin oxide (SnO₂), strontium titanate (SrTiO₃), tungsten oxide (WO₃), bismuth oxide (Bi₂O₃) and iron oxide (Fe₂O₃) that are known as photo-semiconductors can be cited. These can be used singularly or in combination of at least two kinds.

In the present embodiment, in particular, titanium dioxide can be preferably used owing to a large band gap, chemical stability, non-toxicity, and easy availability. There are two types of titanium dioxide, anatase type and rutile type, and both can be used in the present embodiment; however, the anatase type titanium dioxide is more preferable. An excitation wavelength of the anatase type titanium dioxide is 380 nm or less.

As such anatase type titanium dioxide, for instance, an anatase titania sol of hydrochloric acid deflocculation type (trade name: STS-02, manufactured by Ishihara Sangyo Kaisha, Ltd., average particle diameter: 7 nm, and trade name: ST-KO1, manufactured by Ishihara Sangyo Kaisha, Ltd.), an anatase titania sol of nitric acid deflocculation type (trade name: TA-15, manufactured by Nissan Chemical Industries Ltd., average particle diameter: 12 nm) and the like can be cited.

The smaller is a particle diameter of the photocatalyst, the better, because a photocatalyst reaction is caused more effectively. It is preferable to use the photocatalyst with an average particle diameter of 50 nm or less, and one having an average particle diameter of 20 nm or less can be particularly preferably used.

The content of the photocatalyst in the photocatalyst-containing vascular cell adhesion layer in this embodiment can be set in the range of 5 to 95% by weight, preferably 10 to 60% by weight, and further preferably 20 to 40% by weight.

Thereby, the vascular cell adhesive material in the region with the energy irradiation of the photocatalyst-containing vascular cell adhesion layer can be decomposed or denatured.

Here, it is preferable that the photocatalyst used in this embodiment has the adhesion-inhibiting properties to a vascular cell by, for example, having high hydrophilic properties or the like. Thereby, when the vascular cell adhesive material is decomposed or the like, the photocatalyst can be used as the vascular cell adhesion-inhibiting material.

(iii) Others

In this embodiment, not only the vascular cell adhesive material and photocatalyst, but also a binder etc. for improving strength, resistance etc. may be contained as necessity in the photocatalyst-containing vascular cell adhesion layer. In the present embodiment, particularly as the binder, at least after the energy irradiation, a material having the vascular cell adhesion-inhibiting properties of inhibiting adhesion to vascular cells is preferably used. This is because the vascular cell adhesion-inhibiting properties of the vascular cell adhesion-inhibiting portion, which is a region irradiated with energy, can thereby be increased. As such a material, one having the vascular cell adhesion-inhibiting properties prior to the energy irradiation or one obtaining the vascular cell adhesion-inhibiting properties by the action of a photocatalyst upon irradiation with energy may be used.

In the present embodiment, a material that becomes to have the vascular cell adhesion-inhibiting properties, particularly by the action of a photocatalyst upon irradiation with energy, is preferably used as a binder. Thereby, in a region prior to the energy irradiation, the adhesion between the vascular cell adhesive material and vascular cells is not inhibited, and only a region where energy is irradiated can be lowered in the adhesive properties to a vascular cell.

As materials that can be used as such a binder, for instance, ones whose main skeleton has such a high bond energy that cannot be decomposed by the photo-excitation of the photocatalyst, and having an organic substituent which can be decomposed by the action of the photocatalyst are preferably used. For instance, the above-mentioned (1) organopolysiloxane that exhibits large strength by hydrolyzing or polycondensating chloro- or alkoxysilane or the like by a sol-gel reaction and the like, and (2) organopolysiloxane and the like in which reactive silicones excellent in the water repellency or oil repellency are crosslinked can be cited.

In the case of the (1), it is preferable to be organopolysiloxanes that are hydrolysis condensates or cohydrolysis condensates of at least one kind of silicon compounds expressed by a general formula:
YnSiX(4−n)
(Here, Y denotes an alkyl group, fluoroalkyl group, vinyl group, amino group, phenyl group, epoxy group or organic group containing the above, and X denotes an alkoxyl group, acetyl group or halogen. “n” is an integer of 0 to 3.).
The number of carbons of the organic group expressed with Y is preferably in the range of 1 to 20, and the alkoxy group expressed with X is preferably a methoxy group, ethoxy group, propoxy group or butoxy group.

As the reactive silicone according to the (2), compounds having a skeleton expressed by a general formula below can be cited.

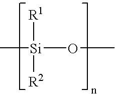

In the above general formula, n denotes an integer of 2 or more, R¹ and R² each represents a substituted or nonsubstituted alkyl group, alkenyl group, aryl group or cyanoalkyl group having 1 to 20 carbons, and a vinyl, phenyl and halogenated phenyl occupy 40% or less by mole ratio to a total mole. Furthermore, one in which R¹ and R² is a methyl group is preferable because the surface energy is the lowest, and a methyl group is preferably contained 60% or more by mole ratio. Still furthermore, a chain terminal or side chain has at least one or more reactive group such as a hydroxyl group in a molecular chain. When the material mentioned above is used, by the action of a photocatalyst upon irradiation with energy, a surface of an energy-irradiated region can be made high in the hydrophilicity. Thereby, the adhesion to vascular cells is inhibited, and the region where energy is irradiated can be made into a region on which the vascular cells do not adhere.

Together with the above-mentioned organopolysiloxanes, a stable organo silicium compound that does not cause a crosslinking reaction, such as dimethylpolysiloxanes, may be blended with a binder.

When the above-mentioned material is used as the vascular cell adhesion-inhibiting material, the contact angle thereof with water is preferably in the range of 15° to 120°, more preferably 20° to 100° before the material is irradiated with energy. According to this, the adhesion of the vascular cell adhesive material to vascular cells is not inhibited.

In the case of irradiating this vascular cell adhesion-inhibiting material with energy, it is preferred that the contact angle thereof with water becomes 10° or less. This range makes it possible to render the material having a high hydrophilicity and low adhesive properties to a vascular cell. Here, the contact angle with water is measured by the above-mentioned method.

In the present embodiment, a decomposition substance or the like that causes such as a change in the wettability of a region where energy is irradiated, thereby lowers the adhesive properties to a vascular cell or that aides such a change may be contained.

As such decomposition substances, for instance, surfactants or the like that are decomposed and the like, by the action of a photocatalyst upon irradiation with energy, to be hydrophilic and the like to result in lowering the adhesive properties to a vascular cell can be cited. Specifically, nonionic surfactants: hydrocarbon based such as respective series of NIKKOL BL, BC, BO, and BB manufactured by Nikko Chemicals Co., Ltd.; and silicone based such as ZONYL FSN and FSO manufacture by Du Pont Kabushiki Kaisha, Surflon S-141 and 145 manufactured by ASAHI GLASS CO., LTD., Megaface F-141 and 144 manufactured by DAINIPPON INK AND CHEMICALS, Inc., FTERGENT F-200 and F-251 manufactured by Neos, UNIDYNE DS-401 and 402 manufactured by DAIKIN INDUSTRIES,Ltd., and Fluorad FC-170 and 176 manufactured by 3M can be cited. Cationic surfactants, anionic surfactants and amphoteric surfactants also can be used.

Other than the surfactants, oligomers and polymers such as polyvinyl alcohol, unsaturated polyester, acrylic resin, polyethylene, diallyl phthalate, ethylene propylene diene monomer, epoxy resin, phenol resin, polyurethane, melamine resin, polycarbonate, polyvinyl chloride, polyamide, polyimide, styrene-butadiene rubber, chloroprene rubber, polypropylene, polybutylene, polystyrene, polyvinyl acetate, nylon, polyester, polybutadiene, polybenzimidazole, polyacrylonitrile, epichlorohydrine, polysulfide, polyisoprene and the like can be cited.

In the present embodiment, such a binder can be preferably contained in the photocatalyst-containing vascular cell adhesion layer, in the range of 5% by weight to 95% by weight, more preferably 40% by weight to 90% by weight, and particularly preferably 60% by weight to 80% by weight.

b. Base Material

Next, the base material used in the present embodiment will be explained. The base material used in the present embodiment is not particularly limited and, for example, an inorganic material such as metal, glass and silicon, or an organic material typified by plastics and the like can be used.

Moreover, the flexibility or the like of the base material can be selected optionally according to the kind, the application or the like of the vascular cell culture patterning substrate. Moreover, the transparency of the base material can be selected optionally according to the kind of the vascular cell culture patterning substrate or the irradiation direction of the energy to be irradiated for decomposing or denaturing the vascular cell adhesive material or the like. For example, in the case the base material has the light-shielding portion or the like, and the energy irradiation is carried out from the base material side or the like, the base material needs to have the transparency.

Here, in this embodiment, a light-shielding portion may be formed in the region, in which the vascular cell adhesion portion is to be formed, on the base material. Thereby, at the time of forming the vascular cell adhesion-inhibiting portion by irradiating the energy to the region in which the vascular cell adhesion-inhibiting portion is to be formed, by irradiating the energy on the entire surface, without the use of a photo mask or the like, the vascular cell adhesive material in the photocatalyst-containing vascular cell adhesion layer can be decomposed or denatured.

The light-shielding portion to be used in this embodiment is not particularly limited as long as it can shield the energy to be irradiated to the vascular cell culture patterning substrate at the time of forming the vascular cell adhesion-inhibiting portion. For example, it may be formed by forming a thin film of a metal such as chromium, in about a 1,000 to 2,000 Å thickness, by a sputtering method, a vacuum deposition method or the like, and then, patterning the thin film. As the patterning method, an ordinary patterning method such as sputtering can be used.

Moreover, it may be formed by a method in which a layer containing light-shielding particles such as carbon particulates, metal oxides, inorganic pigments and organic pigments in a resin binder is formed in a pattern. As the resin binders that can be used, a polyimide resin, acrylic resin, epoxy resin, polyacrylamide, polyvinyl alcohol, gelatin, casein, cellulose and the like can be used singularly or in combination of two or more kinds. Furthermore, a photosensitive resin and an O/W emulsion type resin composition such as emulsified reactive silicone can be used. A thickness of such the resinous light-shielding portion can be set in the range of 0.5 to 10 μm. As a method for patterning such the resinous light-shielding portion, methods such as a photolithography method and a printing method that are generally used can be used.

c. Method for Forming a Vascular Cell Adhesion-inhibiting Portion

Next, the method for forming a vascular cell adhesion-inhibiting portion in this embodiment will be explained. In this embodiment, for example as shown in FIG. 2, by irradiating the energy 6 in a pattern of avascular cell adhesion-inhibiting portion to be formed, by using for example a photo mask 5 or the like, to the photocatalyst-containing vascular cell adhesion layer 4 formed on the base material 1 (FIG. 2A), the vascular cell adhesive material in the photocatalyst-containing vascular cell adhesion layer 4, of the region irradiated with the energy, is decomposed or denatured. Thus, a vascular cell adhesion-inhibiting portion 7 having the adhesion-inhibiting properties to a vascular cell can be formed (FIG. 2B). At the time, the vascular cell adhesion-inhibiting portion contains the photocatalyst and the decomposed product or the denatured product of the vascular cell adhesive material, that is, the vascular cell adhesion-inhibiting material or the like.

The energy irradiation (exposure) mentioned in this embodiment is a concept that includes all energy ray irradiation that can decompose or denature the vascular cell adhesive material by the action of a photocatalyst upon irradiation with energy, and is not limited to light irradiation.

Normally, as energy used in such energy irradiation, ultraviolet light of 400 nm or less can be listed. This is because, as mentioned above, the photocatalyst that is preferably used as a photocatalyst is titanium dioxide, and as energy that activates a photocatalyst action by the titanium oxide, light having the above-mentioned wavelength is preferable.

As a light source that can be used in such energy irradiation, a mercury lamp, metal halide lamp, xenon lamp, excimer lamp and other various kinds of light sources can be cited.

Other than the method in which pattern irradiation is carried out via a photomask by using the above-mentioned light source, a method of carrying out drawing irradiation in a pattern by using laser such as excimer, YAG and the like can be used. Furthermore, as mentioned above, when the base material has the light-shielding portion in a pattern same as that of the vascular cell adhesion portion, energy can be irradiated over the entire surface from the base material side. In this case, there are advantages in that there are no needs of the photomask and the like and a process of positional alignment and the like are also not necessary.

An amount of irradiation of energy at the energy irradiation is an amount of irradiation necessary for decomposing or denaturing the vascular cell adhesive material by the action of the photocatalyst.

At this time, by irradiating a layer containing the photocatalyst, with energy, while heating, the sensitivity can be raised; accordingly, it is preferable in that the vascular cell adhesive material can be efficiently decomposed or denatured. Specifically, it is preferable to heat in the range of 30° C. to 80° C.

The energy irradiation that is carried out via a photomask in this embodiment, when the above-mentioned base material is transparent, may be carried out from either direction of the base material side or a photocatalyst-containing vascular cell adhesion layer side. On the other hand, when the base material is opaque, it is necessary to irradiate energy from a photocatalyst-containing vascular cell adhesion layer side.

In the case of manufacturing the vascular cell culture patterning substrate according to this embodiment and manufacturing a blood vessel by adhering the vascular cells to the vascular cell adhesion portion, by irradiating the energy to the vascular cell adhesion-inhibiting portion using the same method as the above-mentioned energy irradiation method, a process of maintaining the vascular cell pattern can be carried out. By an action of a photocatalyst upon irradiation with energy or the like, the vascular cells adhered on the vascular cell adhesion-inhibiting portion can be removed or the like so that the vascular cells can be cultured in a highly precise pattern. The energy may be irradiated throughout the blood vessel formation, or it may be carried out optionally as needed, or the like.

### (2) SECOND EMBODIMENT

Next, the second embodiment is a vascular cell culture patterning substrate, wherein: a photocatalyst-containing layer and a vascular cell adhesion layer are formed on the base material; the photocatalyst-containing layer contains at least a photocatalyst; the vascular cell adhesion layer contains a vascular cell adhesive material which has adhesive properties to a vascular cell, and is to be decomposed or denatured by an action of a photocatalyst upon irradiation with energy; and in the vascular cell adhesion-inhibiting portion, the vascular cell adhesive material have been decomposed or denatured by an action of a photocatalyst upon irradiation with energy.

In this embodiment, since the vascular cell adhesion layer is formed on the photocatalyst containing layer, by irradiating the energy to the region on which the vascular cell adhesion-inhibiting portion is formed, the vascular cell adhesive material in the vascular cell adhesion layer can be decomposed or denatured by the action of the photocatalyst in the adjacent photocatalyst-containing layer so that the adhesive properties to a vascular cell, of the region, can be lowered so as to enable the formation of the vascular cell adhesion-inhibiting portion having the vascular cell adhesion-inhibiting properties. At the time, for example in the case the vascular cell adhesive material is decomposed by an action of a photocatalyst upon irradiation with energy, the vascular cell adhesion-inhibiting portion contains a small amount of the vascular cell adhesive material, or it contains the decomposed product of the vascular cell adhesive material or the like, or the vascular cell adhesion layer is completely decomposed and removed so as to expose the photocatalyst-containing layer or the like.

Moreover, in the case the vascular cell adhesive material is denatured by an action of a photocatalyst upon irradiation with energy, the vascular cell adhesion-inhibiting portion contains the denatured product thereof or the like.

Hereinafter, each configuration of this embodiment will be explained. Since the base material used in this embodiment, and the method for forming the vascular cell adhesion-inhibiting portion in this embodiment are same as those described in the first embodiment, the description thereof is omitted here.

Also in the case of manufacturing the vascular cell culture patterning substrate according to the present embodiment and manufacturing a blood vessel by adhering the vascular cells to the vascular cell adhesion portion, a process of maintaining the vascular cell pattern can be carried out by irradiating the energy to the vascular cell adhesion-inhibiting portion using the same method as the above-mentioned energy irradiation method.

a. Vascular Cell Adhesion Layer

First, the vascular cell adhesion layer used in this embodiment will be explained. The vascular cell adhesion layer used in this embodiment is a layer contains at least a vascular cell adhesive material having the adhesive properties to a vascular cell so that a layer commonly used as a layer having the adhesive properties to a vascular cell can be used.

As to the specific vascular cell adhesive material, since the same vascular cell adhesive material used in the photocatalyst-containing vascular cell adhesion layer explained in the first embodiment can be used, the detailed description thereof is omitted here. Moreover, it is preferable that the vascular cell adhesion layer in this embodiment also contains the material having the vascular cell adhesion-inhibiting properties explained for the photocatalyst-containing vascular cell adhesion layer of the first embodiment. Thereby, the vascular cell adhesion-inhibiting properties of the vascular cell adhesion-inhibiting portion, which is the region irradiated with the energy, can further be improved.

Moreover, such a vascular cell adhesion layer can be formed by coating a vascular cell adhesion layer forming coating solution, containing the vascular cell adhesive material, by a common coating method or the like. Since it can be same as the method for forming the photocatalyst-containing vascular cell adhesion layer of the first embodiment, the description thereof is omitted here. Moreover, a commonly used adsorption method can be used as well.

The film thickness of the vascular cell adhesion layer can be selected optionally according to the kind of the vascular cell culture patterning substrate or the like. It is in general about 0.001 μm to 1.0 μm, in particular, about 0.05 μm to 0.3 μm.

b. Photocatalyst-containing Layer

Next, the photocatalyst-containing layer used in this embodiment will be explained. The photocatalyst-containing layer used in this embodiment is not particularly limited as long as it is a layer containing at least a photocatalyst. It may be a layer containing only a photocatalyst, or it may be a layer containing other components such as a binder or the like.

The photocatalyst used in this embodiment can be same as those used in the photocatalyst-containing vascular cell adhesion layer in the first embodiment. Also in this embodiment, it is particularly preferable to use a titanium oxide.

The photocatalyst-containing layer consisting of a photocatalyst only is advantageous in costs because the efficiency of decomposing or denaturing the vascular cell adhesive material in the vascular cell adhesion layer is improved to reduce the treatment time. On the other hand, use of the photocatalyst-containing layer comprising a photocatalyst and a binder is advantageous in that the photocatalyst-containing layer can be easily formed.

An example of the method for forming the photocatalyst-containing layer made only of a photocatalyst may be a vacuum film-forming method such as sputtering, CVD or vacuum vapor deposition. The formation of the photocatalyst-containing layer by the vacuum film-forming method makes it possible to render the layer a homogeneous photocatalyst-containing layer made only of a photocatalyst. Thereby, the vascular cell adhesive material can be decomposed or denatured homogeneously. At the same time, since the layer is made only of a photocatalyst, the vascular cell adhesive material can be decomposed or denatured more effectively, as compared with the case of using a binder.

Another example of the method for forming the photocatalyst-containing layer made only of a photocatalyst, is the following method: for example, in the case that the photocatalyst is titanium dioxide, amorphous titania is formed on the base material, and then, calcinating so as to phase-change the titania to crystalline titania. The amorphous titania used in this case can be obtained, for example, by hydrolysis or dehydration condensation of an inorganic salt of titanium, such as titanium tetrachloride or titanium sulfate, or hydrolysis or dehydration condensation of an organic titanium compound, such as tetraethoxytitanium, tetraisopropoxytitanium, tetra-n-propoxytitanium, tetrabutoxytitanium or tetramethoxytitanium, in the presence of an acid. Next, the resultant is calcinated at 400° C. to 500° C. so as to be denatured to anatase type titania, and calcinated at 600° C. to 700° C. so as to be denatured to rutile type titania.

In the case of using a binder, the binder preferably having a high bonding energy, wherein its main skeleton is not decomposed by photoexcitation of the photocatalyst. Examples of such a binder include the organopolysiloxanes described in the above-mentioned item “Vascular cell adhesion layer”.

In the case of using such an organopolysiloxane as the binder, the photocatalyst-containing layer can be formed by dispersing a photocatalyst, the organopolysiloxane as the binder, and optional additives if needed into a solvent to prepare a coating solution, and coating this coating solution onto the base material. The used solvent is preferably an alcoholic based organic solvent such as ethanol or isopropanol. The coating can be performed by a known coating method such as spin coating, spray coating, dip coating, roll coating and bead coating. When the coating solution contains an ultraviolet curable component as the binder, the photocatalyst-containing layer can be formed by curing treatment through the irradiation of ultraviolet rays.

As the binder, an amorphous silica precursor can be used. This amorphous silica precursor is preferably: a silicon compound represented by the general formula SiX₄, wherein X are a halogen, a methoxy group, an ethoxy group, an acetyl group or the like; a silanol which is a hydrolyzate thereof; or a polysiloxane having an average molecular weight of 3000 or less.

Specific examples thereof include such as tetraethoxysilane, tetraisopropoxysilane, tetra-n-propoxysilane, tetrabutoxysilane, and tetramethoxysilane. In this case, the photocatalyst-containing layer can be formed by: dispersing the amorphous silica precursor and particles of a photocatalyst homogeneously into a non-aqueous solvent; hydrolyzing with water content in the air to form a silanol onto a transparent base material; and then subjecting to dehydration polycondensation at room temperature. When the dehydration polycondensation of the silanol is performed at 100° C. or higher, the polymerization degree of the silanol increases so that the strength of the film surface can be improved. A single kind or two or more kinds of this binding agent may be used.

The content of the photocatalyst in the photocatalyst-containing layer can be set in the range of 5 to 60% by weight, preferably in the range of 20 to 40% by weight. The thickness of the photocatalyst-containing layer is preferably in the range of 0.05 to 10 μm.

Besides the above-mentioned photocatalyst and binder, the surfactant and so on used in the above-mentioned vascular cell adhesion layer can be incorporated into the photocatalyst-containing layer.

Here, in the case the vascular cell adhesion layer is a layer to be completely decomposed by an action of a photocatalyst upon irradiation with energy, since the photocatalyst-containing layer is exposed in the region to be the vascular cell adhesion-inhibiting portion, the vascular cell adhesion-inhibiting material should be contained in the photocatalyst-containing layer. In this case, the above-mentioned vascular cell adhesion-inhibiting material may be contained in the photocatalyst-containing layer, or a photocatalyst having a high hydrophilic properties or the like may be used as the vascular cell adhesion-inhibiting material.

Moreover, in this embodiment, as mentioned above, a light-shielding portion may be formed on the photocatalyst containing layer. Thereby, in the case the energy is irradiated onto the entire surface of the vascular cell adhesion layer, the vascular cell adhesive material contained in the vascular cell adhesion layer, in the region other than the region provided with the light shielding portion, can be decomposed or denatured, without exciting the photocatalyst on the region provided with the light shielding portion. Moreover, in this case, since the photocatalyst in the region provided with the light-shielding portion is not excited, the energy irradiation direction is not particularly limited, and thus it is advantageous.

As the light-shielding portion, since those explained in the first embodiment can be used, the detailed description thereof is omitted here.

### (3) THIRD EMBODIMENT

Moreover, the third embodiment is the vascular cell culture patterning substrate, wherein: at least a vascular cell adhesion layer is formed on the base material; the vascular cell adhesion layer contains a vascular cell adhesive material which has adhesive properties to a vascular cell, and is to be decomposed or denatured by an action of a photocatalyst upon irradiation with energy; and in the vascular cell adhesion-inhibiting portion, the vascular cell adhesive material have been decomposed or denatured by an action of a photocatalyst upon irradiation with energy.

In this embodiment, by disposing the vascular cell adhesion layer and the photocatalyst-containing layer facing to each other, and irradiating the energy in a pattern of the vascular cell adhesion-inhibiting portion to be formed, the vascular cell adhesive material in the vascular cell adhesion layer is decomposed or denatured by the action of the photocatalyst in the photocatalyst-containing layer so that the vascular cell adhesion-inhibiting portion can be formed.

Hereinafter, the photocatalyst-containing layer side substrate used in this embodiment, and the method for forming a vascular cell adhesion-inhibiting portion using the photocatalyst-containing layer side substrate will be explained. Since the vascular cell adhesion layer used in this embodiment is same as that used in the second embodiment, the description thereof is omitted here. Moreover, in the case the vascular cell adhesion layer is a layer to be completely decomposed by an action of a photocatalyst upon irradiation with energy, since the region to be the vascular cell adhesion-inhibiting portion has the base material exposed, the vascular cell adhesion-inhibiting material as mentioned above needs to be contained in the base material.

a. Photocatalyst-containing Layer Side Substrate

First, the photocatalyst-containing layer side substrate, comprising a photocatalyst-containing layer containing a photocatalyst, used in this embodiment is described. The photocatalyst-containing layer side substrate used in this embodiment usually comprises a photocatalyst-containing layer containing a photocatalyst, which usually comprises a base body and a photocatalyst-containing layer formed on the base body. This photocatalyst-containing layer side substrate may have, for example, photocatalyst-containing layer side light-shielding portion formed in a pattern form, a primer layer or the like. The following will describe each constituent of the photocatalyst-containing layer side substrate used in this embodiment.

(i) Photocatalyst-containing Layer

First, the photocatalyst-containing layer used in the photocatalyst-containing layer side substrate is described. The photocatalyst-containing layer used in this embodiment is not particularly limited insofar as the layer is constituted such that the photocatalyst in the photocatalyst-containing layer can cause the decomposition or denaturation of the vascular cell adhesive material in the adjacent vascular cell adhesion layer. The photocatalyst-containing layer may be composed of a photocatalyst and a binder or may be made of a photocatalyst only. The property of the surface thereof maybe lyophilic or repellent to liquid.

The photocatalyst-containing layer used in this embodiment may be formed on the whole surface of a base body, or as shown in, for example, FIG. 3, a photocatalyst-containing layer 12 may be formed in a pattern form on a base body 11.

By forming the photocatalyst-containing layer in a pattern accordingly, at the time of irradiating the energy to form the vascular cell adhesion-inhibiting portion, without the need of the pattern irradiation using a photo mask or the like, by the entire surface irradiation, the vascular cell adhesion-inhibiting portion, in which the vascular cell adhesive material contained in the vascular cell adhesion layer is decomposed or denatured, can be formed.

The patterning method for the photocatalyst-containing layer is not particularly limited. It can be carried out by, for example, the photolithography method or the like.

Moreover, since the vascular cell adhesive material only in the portion of the vascular cell adhesion layer actually facing the photocatalyst-containing layer is decomposed or denatured, the energy irradiation direction may be of any direction as long as the energy is irradiated to the portion where the photocatalyst-containing layer and the vascular cell adhesion layer face to each other. Furthermore, there is an advantage that irradiated energy is not particularly limited to parallel ones such as a parallel beam, etc.

Here, since the photocatalyst-containing layer used in this embodiment is same as the photocatalyst-containing layer explained in the second embodiment, the detailed the description thereof is omitted here.

(ii) Base Body

The following will describe the base body used in the photocatalyst-containing layer side substrate. Usually, the photocatalyst-containing layer side substrate comprises at least a base body and a photocatalyst-containing layer formed on the base body. In this case, the material which constitutes the base body to be used is appropriately selected depending on the direction of energy irradiation which will be detailed later, necessity of the resulting pattern-forming body to be transparency, or other factors.

The base body used in this embodiment may be a member having flexibility such as a resin film, or may be a member having no flexibility such as a glass substrate. This is appropriately selected depending on the method of the energy irradiation.

An anchor layer may be formed on the base body in order to improve the adhesion between the surface of the base body and the photocatalyst-containing layer. The anchor layer may be made of, for example, a silane based or titanium based coupling agent.

(iii) Photocatalyst-Containing Layer Side Light-Shielding Portion

As the photocatalyst-containing layer side substrate in this embodiment, a photocatalyst-containing layer side substrate provided with pattern-formed photocatalyst-containing layer side light-shielding portion can be used. When the photocatalyst-containing layer side substrate having photocatalyst-containing layer side light-shielding portion is used in this way, at the time of irradiating energy, it is not necessary to use any photomask or to carry out drawing irradiation with a laser light. Since alignment of the photomask and the photocatalyst-containing layer side substrate is not necessary, process can be made simple. Further, since expensive device for drawing irradiation is also not necessary, it is advantageous in costs.

Such a photocatalyst-containing layer side substrate having photocatalyst-containing layer side light-shielding portion can be classified into the following two embodiments, depending on the position where the photocatalyst-containing layer side light-shielding portion is formed.

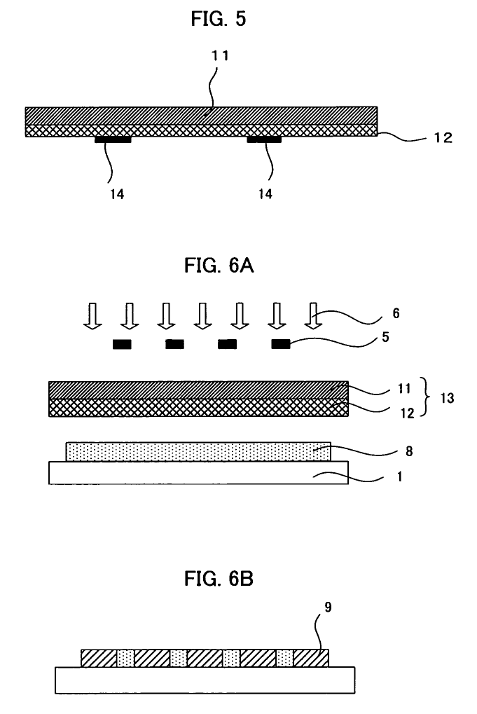

One of them is an embodiment, as shown in FIG. 4 for example, wherein photocatalyst-containing layer side light-shielding portion 14 is formed on a base body 11, and a photocatalyst-containing layer 12 is formed on the photocatalyst-containing layer side light-shielding portion 14 to obtain the photocatalyst-containing layer side substrate. The other example is an embodiment, as shown in FIG. 5 for example, wherein a photocatalyst-containing layer 12 is formed on a base body 11, and photocatalyst-containing layer side light-shielding portion 14 is formed thereon to obtain the photocatalyst-containing layer side substrate.

In any one of these embodiments, since the photocatalyst-containing layer side light-shielding portion is arranged near the region where the photocatalyst-containing layer and the vascular cell adhesion layer are arranged, the effect of energy-scattering in the base body or the like can be made smaller than in the case of using a photomask. Accordingly, irradiation of energy in a pattern can be more precisely attained.

In this embodiment, in the case of the embodiment wherein the photocatalyst-containing layer side light-shielding portion 14 is formed on a photocatalyst-containing layer 12 as shown in FIG. 5, there is an advantage that at the time of arranging the photocatalyst-containing layer and the vascular cell adhesion layer in a predetermined position, the photocatalyst-containing layer side light-shielding portion can be used as a spacer for making the interval constant, by making the film thickness of the photocatalyst-containing layer side light-shielding portion corresponding to the width of the interval between the two layers.

In other words, when the photocatalyst-containing layer and the vascular cell adhesion layer are arranged so as to be facing each other at a predetermined interval, by arranging the photocatalyst-containing layer side light-shielding portion and the vascular cell adhesion layer in close contact to each other, the dimension of the predetermined interval can be made precise. When energy is irradiated in this state, vascular cell adhesion-inhibiting portion can be formed with a good precision since vascular cell adhesive material in the vascular cell adhesion layer, inside the region where the vascular cell adhesion layer and the light-shielding portion are in contact, is not decomposed or denatured.

The method for forming such photocatalyst-containing layer side light-shielding portion is not particularly limited, and may be appropriately selected in accordance with the property of the surface on which the photocatalyst-containing layer side light-shielding portion is to be formed, shielding ability against the required energy, and others. Since the light-shielding portion may be the same as the light-shielding portion provided on the base material that is described in the first embodiment. Thus, the detailed description thereof is omitted herein.

The above has described two cases, wherein the photocatalyst-containing layer side light-shielding portion is formed in between the base body and the photocatalyst-containing layer and wherein it is formed on the surface of the photocatalyst-containing layer. Besides, the photocatalyst-containing layer side light-shielding portion may be formed on the base body surface of the side on which the photocatalyst-containing layer is not formed. In this embodiment, for example, a photomask can be made in close contact to this surface to such a degree that the photomask in removable. Thus, such method can be preferably used for the case that the pattern of the vascular cell adhesion auxiliary portions is changed for every small lot.

(iv) Primer Layer

The following will describe a primer layer used in the photocatalyst-containing layer side substrate of this embodiment. In this embodiment, when photocatalyst-containing layer side light-shielding portion is formed into a pattern on a base body and a photocatalyst-containing layer is formed thereon so as to prepare a photocatalyst-containing layer side substrate described above, a primer layer may be formed in between the photocatalyst-containing layer side light-shielding portion and the photocatalyst-containing layer.

The effect and function of this primer layer are not necessarily clear, but would be as follows: by forming the primer layer in between the photocatalyst-containing layer side light-shielding portion and the photocatalyst-containing layer, the primer layer is assumed to exhibit a function of preventing the diffusion of impurities from the photocatalyst-containing layer side light-shielding portion and openings present between the photocatalyst-containing layer side light-shielding portions, in particular, residues generated when the photocatalyst-containing layer side light-shielding portion is patterned, or metal or metal ion impurities; the impurities being factors for blocking the decomposition or denaturation of the vascular cell adhesive material by action of the photocatalyst. Accordingly, by forming the primer layer, it is possible to process the decomposition or denaturation of the vascular cell adhesive material with high sensitivity, so as to yield vascular cell adhesion-inhibiting portion which are highly precisely formed.

The primer layer in this embodiment is a layer for preventing the action of the photocatalyst from being affected by the impurities present in not only the photocatalyst-containing layer side light-shielding portion but also in the openings formed between the photocatalyst-containing layer side light-shielding portions. It is therefore preferred to form the primer layer over the entire surface of the photocatalyst-containing layer side light-shielding portion including the openings.

The primer layer in this embodiment is not particularly limited insofar as the primer layer is formed not to bring the photocatalyst-containing layer side light-shielding portion and the photocatalyst-containing layer of the photocatalyst-containing layer side substrate into contact with each other.

A material that forms the primer layer, though not particularly limited, is preferably an inorganic material that is not likely to be decomposed by the action of the photocatalyst. Specifically, amorphous silica can be cited. When such amorphous silica is used, a precursor of the amorphous silica is preferably a silicon compound that is represented by a general formula, SiX₄, wherein X being halogen, methoxy group, ethoxy group, acetyl group or the like; silanol that is a hydrolysate thereof, or polysiloxane having an average molecular weight of 3000 or less.

A film thickness of the primer layer is preferably in the range of 0.001 μm to 1 μm and particularly preferably in the range of 0.001 μm to 0.1 μm.

b. Method for Forming Vascular Cell Adhesion-inhibiting Portion

Hereinafter, the method for forming the vascular cell adhesion-inhibiting portion in this embodiment is described. In this embodiment, for example as shown in FIG. 6, a vascular cell adhesion layer 8 formed on a base material 1, and a photocatalyst-containing layer 12 of a photocatalyst-containing layer side substrate 13, are arranged with a predetermined space and irradiated with energy 6 from a predetermined direction, for example, via photomask 5 or the like (FIG. 6A). The vascular cell adhesive material in the region irradiated with energy is thereby decomposed or denatured, thus forming the vascular cell adhesion-inhibiting portion 9 having vascular cell adhesion-inhibiting properties (FIG. 6B). In this case, when the vascular cell adhesive material is decomposed for example by an action of a photocatalyst upon irradiation with energy, the vascular cell adhesion-inhibiting portion contains a small amount of the vascular cell adhesive material or contains decomposed products of the vascular cell adhesive material. Otherwise, the vascular cell adhesion layer is completely decomposed and removed to expose the base material. When the vascular cell adhesive material is denatured by an action of a photocatalyst upon irradiation with energy, its denatured products are contained in the vascular cell adhesion-inhibiting portion.

The above-mentioned wording “arranging” means that the layers are arranged in the state that the action of the photocatalyst can substantially work to the surface of the vascular cell adhesion layer, and include not only the state that the two layers actually contact each other, but also the state that the photocatalyst-containing layer and the vascular cell adhesion layer are arranged at a predetermined interval. The dimension of the interval is preferably 200 μm or less.

In this embodiment, the dimension of the above-mentioned interval is more preferably in the range of 0.2 μm to 10 μm, even more preferably in the range of 1 μm to 5 μm, since the precision of the pattern to be obtained becomes very good and the sensitivity of the photocatalyst becomes high so as to make good efficiency of the decomposition or denaturation of the vascular cell adhesive material in the vascular cell adhesion layer. This range of the interval dimension is particularly effective for the vascular cell adhesion layer which is small in area, wherein the interval dimension can be controlled with a high precision.

Meanwhile, in the case of treating the vascular cell adhesion layer having large area, for example, 300 mm×300 mm or more in size, it is very difficult to make a fine interval as described above in between the photocatalyst-containing layer side substrate and the vascular cell adhesion layer without contacting each other. Accordingly, when the vascular cell adhesion layer has a relatively large area, the interval dimension is preferably in the range of 10 to 100 μm, more preferably in the range of 50 to 75 μm. By setting the interval dimension in the above range, problems will not occur that: deterioration of patterning precision, such as blurring of the pattern; or the sensitivity of the photocatalyst deteriorates so that the efficiency of decomposing or denaturing the vascular cell adhesive material is also deteriorated. Further, there is an advantageous effect that the vascular cell adhesive material is not unevenly decomposed or denatured.

When energy is irradiated onto the vascular cell adhesion layer having a relatively large area as described above, the dimension of the interval, in a unit for positioning the photocatalyst-containing layer side substrate and the vascular cell adhesion layer inside the energy irradiating device, is preferably set in the range of 10 μm to 200 μm, more preferably in the range of 25 μm to 75 μm. The setting of the interval dimension value into this range makes it possible to arrange the photocatalyst-containing layer side substrate and the vascular cell adhesion layer without causing a large deterioration of patterning precision or of sensitivity of the photocatalyst, or bringing the substrate and the layer into contact with each other.

When the photocatalyst-containing layer and the surface of the vascular cell adhesion layer are arranged at a predetermined interval as described above, active oxygen species generated from oxygen and water by action of the photocatalyst can easily be released. In other words, if the interval between the photocatalyst-containing layer and the vascular cell adhesion layer is made narrower than the above-mentioned range, the active oxygen species are not easily released, so as to make the rate for decomposing or denaturing the vascular cell adhesive material unfavorably small. If the two layers are arranged at an interval larger than the above-mentioned range, the generated active oxygen species do not reach the vascular cell adhesion layer easily. In this case also, the rate for decomposing or denaturing the vascular cell adhesive material becomes unfavorably small.

The method for arranging the photocatalyst-containing layer and the vascular cell adhesion layer to make such a very small interval evenly therebetween is, for example, a method of using spacers. The use of the spacers in this way makes it possible to make an even interval. At the same time, the action of the photocatalyst does not work onto the surface of the vascular cell adhesion layer in the regions which the spacers contact. Therefore, when the spacers are rendered to have a pattern similar to that of the vascular cell adhesion portions, the vascular cell adhesive material only inside regions where no spacers are formed can be decomposed or denatured so that highly precise vascular cell adhesion-inhibiting portions can be formed. The use of the spacers also makes it possible that the active oxygen species generated by action of the photocatalyst reach the surface of the vascular cell adhesion layer, without diffusing, at a high concentration. Accordingly, highly precise vascular cell adhesion-inhibiting portion can be effectively formed.

In this embodiment, it is sufficient that such an arrangement state of the photocatalyst-containing layer side substrate is maintained only during the irradiation of energy.

The energy irradiation (exposure) mentioned in this embodiment is a concept that includes all energy ray irradiation that can decompose or denature the vascular cell adhesive material by the action of a photocatalyst upon irradiation with energy, and is not limited to light irradiation.

Here, since the kind, etc. of the energy to be irradiated in this embodiment is same as that explained in the first embodiment, the detailed description thereof is omitted here.

The energy irradiation that is carried out via a photomask in this embodiment, when the above-mentioned base material is transparent, may be carried out from either direction of the base material side or a photocatalyst-containing layer side substrate. On the other hand, when the base material is opaque, it is necessary to irradiate energy from the side of the photocatalyst-containing layer side substrate.

Moreover, also in the case of manufacturing a vascular cell culture patterning substrate according to this embodiment and manufacturing a blood vessel by adhering the vascular cells to the vascular cell adhesion portion, a process of maintaining the vascular cell pattern can be carried out by irradiating the energy to the vascular cell adhesion-inhibiting portion using the photocatalyst-containing layer side substrate. By an action of a photocatalyst upon irradiation with energy or the like, the vascular cells adhered on the vascular cell adhesion-inhibiting portion can be removed or the like so that the vascular cells can be cultured in a highly precise pattern.

B. Method for Manufacturing Blood Vessel

Now, the method for manufacturing a blood vessel of the present invention is described. The method for manufacturing blood vessels of the present invention is a method in which a blood vessel is manufactured by culturing vascular cells by using the above-described vascular cell culture patterning substrate.

In the present invention, by culturing or organizing the vascular cells using the above-mentioned vascular cell culture patterning substrate, the adhesion of the vascular cells to the vascular cell adhesion-inhibiting portion can be inhibited during the culturing or organization of the vascular cells. Therefore, for example, adhesion of the vascular cells adhered on the vascular cell adhesion portion and the vascular cells adhered on the vascular cell adhesion-inhibiting portion to one another, or bonding of the cell pseudopods generated from the vascular cells adhered between the adjacent vascular cell adhesion portions can be inhibited. Thereby, adhesion of the adjacent blood vessels, and rupture of the blood vessels due to the rupture can be prevented so that the blood vessels can be formed in a purposed shape. Moreover, in the present invention, since the distance between the formed plural blood vessels can be made relatively short, at the time of constructing the artificial tissues using the blood vessels, supply of the oxygen or nutrition through the blood vessels to the other cells in between the blood vessels, transportation of wastes produced by the other cells in between the blood cells, or the like is made possible.

The vascular cell culture patterning substrate is the same as described above so that its detailed description is omitted herein, and the vascular cells used in the present invention are described.

The vascular cells used in the present invention are vascular cells which form a blood vessel by being cultured. It refers to vascular endothelial cells, pericytes, smooth muscle cells, vascular endothelial precursor cells and smooth muscle precursor cells derived from organisms, particularly animals. Particularly, it refers to vascular endothelial cells etc. Plural kinds of cells can be co-cultured such as co-culture of vascular endothelial cells and pericytes or co-culture of endothelial cells and smooth muscle cells.

To form blood vessels, when culturing the vascular cells by adhering them on the vascular cell adhesion portion, it is effective to apply shearing stress in uniaxial direction in the same direction as the line pattern of the vascular cell adhesion portion. The adhered form of the vascular cells can thereby become long and thin spindle-shaped, and the respective vascular cells can adhere to one another in such a state that they seem oriented in the above-mentioned uniaxial direction. To form blood vessels, it is important that the vascular cells are adhered in a confluent state such that the vascular cells are adhered in a thin and long form and the vascular cells are directed to the same direction. The method for applying shear stress in the uniaxial direction includes: a method in which the vascular cells are cultured by placing a culture dish on a shaker or a shaking apparatus; and a method in which the vascular cells are cultured while streaming culture liquid in one direction. To form a blood vessel of 5000 μm or more in width, shearing stress in uniaxial direction is essential.

Usually, a blood vessel is obtained by forming the vascular cells in an objective pattern on the vascular cell adhesion portion, and then, adding, to a medium, growth factors such as bFGF and VEGF promoting vascularization of vascular cells. It is estimated that, by stimulation from the growth factors, proliferation of the vascular cells is terminated and differentiated so as to be blood vessels. As the medium for vascularization of vascular cells adhered in a confluent state to the vascular cell adhesion portion, not only a liquid medium containing the growth factor, but also a gelled medium containing the above-described growth factor or a combination of gelled and liquid mediums containing the growth factor can be used. As the gelled medium, collagen, fibrin gel, Matrigel (trade name) or synthetic peptide hydrogel can be used.

The present invention is not limited to the above mentioned embodiments. The above mentioned embodiments are merely examples, and any one having the substantially same configuration and the same effects, or equivalent thereof, as the technological idea disclosed in the claims of the present invention is included in the technological scope of the present invention.

### EXAMPLES

Hereinafter, with reference to the examples, the present invention will be explained further specifically.

### Example 1

[Preparation of a Vascular Cell Culture Patterning Substrate]

(Formation of a Patterning Substrate Having a Light-shielding Layer and a Vascular Cell Adhesion Layer)

A metal light-shielding portion was formed on a glass substrate, to form a 5 inch square quartz photomask, so as to be a stripe pattern of the metal light-shielding portion as a vascular cell adhesion portion of 40 μm, and a glass portion as a vascular cell adhesion-inhibiting portion of 300 μm.

Then, 30 g of an isopropyl alcohol, 3 g of a trimethoxy methyl silane TSL8114 (GE Toshiba silicones), and 20 g of a photocatalyst inorganic coating agent ST-K03 (Ishihara Sangyo Kaisha, Ltd.) were mixed and agitated at 100° C. for 20 minutes. It was diluted 3-fold with an isopropyl alcohol so as to provide a photocatalyst-containing layer composition. By coating the photocatalyst-containing layer composition to the rear side of the quartz photomask provided with the light-shielding portion by a spin coater, and carrying out a drying process at 150° C. for 10 minutes, a transparent photocatalyst-containing layer was formed.

Then, 0.7 g of an alkyl silane LS-5258 (Shin-Etsu Chemical Co., Ltd.), 5.0 g of an organosilane TSL-8114 (GE Toshiba Silicones) as a binder, and 2.36 g of a 0.005N hydrochloric acid were mixed and agitated for 24 hours. By diluting the solution 100-fold with an isopropyl alcohol, coating the solution onto the photocatalyst layer by the spin coating method, and furthermore, drying at a 150° C. temperature for 10 minutes so as to promote the hydrolysis and polycondensation reaction, a patterning substrate having a 0.2 μm thickness vascular cell adhesion layer was obtained.

(Patterning of a Patterning Substrate)

By exposing to the ultraviolet ray with a 15 J/cm² energy amount, using a mercury lamp, from the light-shielding portion side of the patterning substrate, a vascular cell culture patterning culture substrate having the vascular cell adhesive surface, which was patterned so as the unexposed portion has the vascular cell adhesive properties and the exposed portion has the vascular cell adhesion-inhibiting properties, was obtained. Then, the vascular cell culture patterning culture substrate was cut into a 15 mm×25 mm size. At the time, it was cut such that the line pattern of the vascular cell adhesion portion matches to the longer axis of the vascular cell culture patterning culture substrate.

(Dissemination of Vascular Cells and Formation of Tissue)

The substrate was dipped in DMEM medium containing 10% bovine fetal serum, and primary human umbilical vein endothelial cells (HUVECs) were disseminated so as to be a concentration of2×10⁵ cells/ml. The cells were cultured at 37° C. in a 5% carbon dioxide atmosphere for 24 hours to allow the vascular cells to adhere to the vascular cell adhesion portion.

When the vascular cells that had adhered to the substrate were observed, it was confirmed that the vascular cells were aligned along all region in the vascular cell adhesion portion, the vascular cells were in an extended form, and there is no contacting of the pseudopods between the vascular cell adhesion portions.

Further, the DMEM medium was exchanged with one containing bFGF (Sigma) at a concentration of 10 ng/ml, culturing was continued at 37° C. in a 5% carbon dioxide atmosphere for 24 hours, and formation of a vascular tissue composed of continuous vascular cells was confirmed.

### Comparative Example 1

[Preparation of a Vascular Cell Culture Patterning Substrate]

(Formation of a Vascular Cell Culture Substrate having a Light-shielding Layer and a Vascular Cell Adhesion Layer)

A metal light-shielding portion was formed on a glass substrate, to form a quartz photomask, so as to be a stripe pattern of the metal light-shielding portion as a vascular cell adhesion portion of 40 μm, and a glass portion as a vascular cell adhesion-inhibiting portion of 300 μm.

Then, 30 g of an isopropyl alcohol, 3 g of a trimethoxy methyl silane TSL8114 (GE Toshiba silicones), and 20 g of a photocatalyst inorganic coating agent ST-K03 (Ishihara Sangyo Kaisha, Ltd.) were mixed and agitated at 100° C. for 20 minutes. It was diluted 3-fold with an isopropyl alcohol so as to provide a photocatalyst-containing layer composition. By coating the photocatalyst-containing layer composition to the rear side of the quartz photomask provided with the light-shielding portion by a spin coater, and carrying out a drying process at 150° C. for 10 minutes, a transparent photocatalyst-containing layer was formed.

Then, as the vascular cell adhesive material, 2 mg of a fibronectin F-4759 (Sigma) was dissolved in 200 ml of pure water. With the photocatalyst containing layer of the quartz photomask having the photocatalyst containing layer facing upward, the same was soaked in the fibronectin solution and left still at 4° C. for 24 hours. Thereafter, by washing with pure water for three times and drying with a nitrogen gas, a patterning substrate, with a photocatalyst containing layer and a vascular cell adhesion layer laminated, was obtained.

(Patterning of a Patterning Substrate)

By exposing to the ultraviolet ray with a 15 J/cm² energy amount, using a mercury lamp, from the light-shielding portion side of the patterning substrate, a vascular cell culture patterning culture substrate having a pattern, wherein the unexposed portion has the vascular cell adhesive properties, and the fibronectin as the vascular cell adhesive material was decomposed in the exposed portion, but not containing the vascular cell adhesion-inhibiting material, was obtained.

[Dissemination of Vascular Cells and Formation of Tissue]

In the same manner as in the example 1, the vascular cells were disseminated and cultured. Although the vascular cells were adhered along the pattern, the alignment properties thereof was poor, and furthermore, the vascular cells were adhered also to the exposed part. Furthermore, organization of the vascular cells were carried out in the same manner as in the example 1, however, continuous vascular tissues were not formed.

### Example 2

[Preparation of a Vascular Cell Culture Patterning Substrate]

(Formation of a Vascular Cell Culture Substrate having a Light-shielding Layer and a Vascular Cell Adhesion Layer)

A metal light-shielding portion was formed on a glass substrate, to form a quartz photomask, so as to be a stripe pattern of the metal light-shielding portion as a vascular cell adhesion portion of 40 μm, and a glass portion as a vascular cell adhesion-inhibiting portion of 300 μm.

Then, 30 g of an isopropyl alcohol, 4 g of a trimethoxy methyl silane TSL8114 (GE Toshiba silicones), 0.5 g of an alkyl silane LS-5258 (Shin-Etsu Chemical Co., Ltd.) and 15 g of a photocatalyst inorganic coating agent ST-K03 (Ishihara Sangyo Kaisha, Ltd.) were mixed and agitated at 100° C. for 20 minutes. It was diluted 10-fold with an isopropyl alcohol so as to provide a photocatalyst-containing vascular cell adhesion layer composition. By coating the photocatalyst-containing vascular cell adhesion layer composition to the rear side of the quartz photomask provided with the light-shielding portion by a spin coater, and carrying out a drying process at 150° C. for 10 minutes, a transparent photocatalyst-containing vascular cell adhesion layer was formed.

(Patterning of a Patterning Substrate)

By exposing to the ultraviolet ray with a 15 J/cm² energy amount, using a mercury lamp, from the light-shielding portion side of the patterning substrate, a vascular cell culture patterning substrate having the vascular cell adhesive surface, which was patterned so as the unexposed portion has the vascular cell adhesive properties and the exposed portion has the vascular cell adhesion-inhibiting properties, was obtained.

[Dissemination of Vascular Cells and Formation of Tissue]

The vascular cells were disseminated on the substrate by the same procedure as in Example 1. The vascular cells that had adhered to the substrate were observed, and it was confirmed that the vascular cells were aligned along all region in the vascular cell culture portion, the vascular cells were in an extended form, and there is no contacting of the pseudopods between the vascular cell adhesion portions. Further, the vascular cells were formed into a tissue by the same procedure as in Example 1, and formation of the vascular tissue composed of continuous vascular cells was confirmed.

### Example 3

[Preparation of a Photocatalyst-containing Layer Side Substrate]

A metal light-shielding portion was formed on a glass substrate, to form a quartz photomask, so as to be a stripe pattern of the metal light-shielding portion as a vascular cell adhesion portion of 40 μm, and a glass portion as a vascular cell adhesion-inhibiting portion of 300 μm.

5 g of a trimethoxy silane TSL8114 (GE Toshiba silicones) and 2.5 g of a 0.5 normal hydrochloric acid were mixed and agitated for 8 hours. It was diluted 10-fold with an isopropyl alcohol so as to provide a primer layer composition. By coating the primer layer composition onto the pattern surface of the photomask by a spin coating method, and drying the substrate at a 150° C. temperature for 10 minutes, a photomask having a primer layer was obtained.

Then, 30 g of an isopropyl alcohol, 3 g of a trimethoxy methyl silane TSL8114 (GE Toshiba Silicones), and 20 g of a photocatalyst inorganic coating agent ST-K03 (Ishihara Sangyo Kaisha, Ltd.) were mixed and agitated at 100° C. for 20 minutes. By diluting the same 3-fold with an isopropyl alcohol, a photocatalyst containing layer composition was provided.

By coating the photocatalyst containing layer composition onto the photomask provided with the primer layer, with a spin coater, and carrying out a drying process at 150° C. for 10 minutes, a photomask having a transparent photocatalyst-containing layer was formed.

[Preparation of a Vascular Cell Culture Patterning Substrate]

5.0 g of an organosilane TSL-8114 (GE Toshiba Silicones), 0.7 g of an alkyl silane LS-5258 (Shin-Etsu Chemical Co., Ltd.) and 2.36 g of a 0.005N hydrochloric acid were mixed and agitated for 24 hours.

By diluting the solution 100-fold with an isopropyl alcohol, coating the same, by the spin coating method, on a soda glass substrate preliminary subjected to an alkaline treatment, and drying the substrate at a 150° C. temperature for 10 minutes, hydrolysis and polycondensation reaction were promoted so as to obtain a substrate having the vascular cell adhesion layer of 0.2 μm film thickness.

(Patterning of the Substrate)

By exposing to the ultraviolet ray with a 15 J/cm² energy amount using a mercury lamp via the photomask, while the vascular cell adhesion layer of the substrate and the photocatalyst-containing layer of the photomask having the photocatalyst containing layer facing with each other, a vascular cell culture substrate having the vascular cell adhesive surface patterned, wherein the unexposed part has the vascular cell adhesive properties and the exposed part has the vascular cell adhesion-inhibiting properties, was obtained.

[Dissemination of Vascular Cells and Formation of Tissue]

The vascular cells were disseminated on the substrate by the same procedure as in Example 1. The vascular cells that had adhered to the substrate were observed, and it was confirmed that the vascular cells were aligned along all region in the vascular cell adhesion portion, the vascular cells were in an extended form, and there is no contacting of the pseudopods between the vascular cell adhesion portions. Further, the vascular cells were formed into a tissue by the same procedure as in Example 1, and formation of the vascular tissue composed of continuous vascular cells was confirmed.

### Example 4

[Preparation of a Photocatalyst-containing Layer Side Substrate]

A metal light-shielding portion was formed on a substrate, to form a quartz photomask, so as to be a stripe pattern of the glass portion as a vascular cell adhesion portion of 120 μm, and a light-shielding portion as a vascular cell adhesion-inhibiting portion of 350 μm. The photocatalyst-containing layer was formed as in Example 3. Thereby, the photocatalyst-containing layer side substrate was formed.

[Preparation of a Vascular Cell Culture Patterning Substrate]

A fluorine based silane coupling agent XC98-B2742 (GE Toshiba Silicones) was diluted 10-fold with an isopropyl alcohol so as to prepare a coating solution. Using this coating solution, a substrate comprising a vascular cell adhesion-inhibiting layer was prepared by the same procedure as in Example 3.

(Patterning of a Substrate)

By disposing the photocatalyst-containing layer side substrate and the substrate provided with the vascular cell adhesion-inhibiting layer as in Example 3, exposing to the ultraviolet ray with a 6 J/cm² energy amount, a vascular cell culture patterning substrate having the vascular cell adhesive surface, which was patterned so as the unexposed portion has the vascular cell adhesion-inhibiting properties and the exposed portion has the vascular cell adhesive properties, was obtained. Then, the vascular cell culture patterning substrate was cut into a 15 mm×25 mm size as in Example 1.

(Dissemination of Vascular Cells and Formation of Tissue)

The substrate was placed on a culture dish, and HUVEC were disseminated so as to be a concentration of 6×10⁵ cells/ml. The culture dish was placed on a shaker, and the cells were cultured for 30 hours, as in Example 1, to allow the vascular cells to adhere to the vascular cell adhesion portion. While culturing, the shaker was operated slowly as a seesaw to allow the medium to flow in the same direction as in the line pattern of the vascular cell adhesion portion.

After culturing for 30 hours, the medium was carefully removed by suction, and then, 0.4 ml Matrigel (Becton Dickinson) containing bFGF (Sigma) at a concentration of 10 ng/ml was given, as a new medium, onto the substrate. The above was gelled, DMEM medium containing 5% fetal bovine serum was added and was cultured. The cells were cultured at 37° C. in an atmosphere of 5% carbon dioxide for 24 hours, and it was confirmed that the vascular cells had formed a continuous vascular tissue.

### Example 5

[Preparation of a Photocatalyst-containing Layer Side Substrate]

A quartz photomask, comprising a light-shielding portion as a vascular cell adhesion-inhibiting portion of 350 μm in width and a vascular cell adhesion portion of 124.5 μm in width having a vascular cell adhesion auxiliary portion, was prepared. The vascular cell adhesion portion had a pattern of opening part/light-shielding portion each of 4.5 μm /25.5 μm, and the pattern of the opening was pattern of the cell adhesion auxiliary portion. Then, the photocatalyst-containing layer was formed as in Example 3. Thereby, the photocatalyst-containing layer side substrate was formed.

[Preparation of a Vascular Cell Culture Patterning Substrate]

A fluorine based silane coupling agent XC98-B2742 (GE Toshiba Silicones) was diluted 10-fold with an isopropyl alcohol so as to prepare a coating solution. Using this coating solution, a substrate comprising a vascular cell adhesion-inhibiting layer was prepared by the same procedure as in Example 3.

(Patterning of a Substrate)

By disposing the photocatalyst-containing layer side substrate and the substrate provided with the vascular cell adhesion-inhibiting layer as in Example 3, exposing to the ultraviolet ray with a 6 J/cm² energy amount, a vascular cell culture patterning substrate having the vascular cell adhesive surface, which was patterned so as the unexposed portion has the vascular cell adhesion-inhibiting properties and the exposed portion has the vascular cell adhesive properties, was obtained. Then, the vascular cell culture patterning substrate was cut into a 15 mm×25 mm size as in Example 1.

(Dissemination of Vascular Cells and Formation of Tissue)

HUVEC were disseminated and formed into a tissue by the same procedure as in Example 1. The vascular cells that had adhered to the substrate were observed, and it was confirmed that the vascular cells were aligned along all region in the vascular cell adhesion portion, the vascular cells were in an extended form, and there is no contacting of the pseudopods between the vascular cell adhesion portions.

1-6. (canceled)

7. A vascular cell culture patterning substrate comprising:
a base material;
a vascular cell adhesion portion formed in at least two substantially parallel lines on the base material, and having adhesive properties to a vascular cell which forms a blood vessel; and
a vascular cell adhesion-inhibiting portion formed in between two adjacent vascular cell adhesion portions on the base material, and inhibiting adhesion to the vascular cell,
wherein the vascular cell adhesion-inhibiting portion contains a vascular cell adhesion-inhibiting material having vascular cell adhesion-inhibiting properties of inhibiting adhesion to the vascular cell.

8. The vascular cell culture patterning substrate according to claim 7, wherein the width of the vascular cell adhesion-inhibiting portion is in the range of 200 μm to 600 μm.

9. The vascular cell culture patterning substrate according to claim 7, wherein a photocatalyst-containing vascular cell adhesion layer is formed on the base material,
the photocatalyst-containing vascular cell adhesion layer contains at least: a photocatalyst; and a vascular cell adhesive material which has adhesive properties to a vascular cell, and is to be decomposed or denatured by an action of a photocatalyst upon irradiabon with energy, and
in the vascular cell adhesion-inhibiting portion, the vascular cell adhesive material have been decomposed or denatured by an action of a photocatalyst upon irradiation with energy.

10. The vascular cell culture patterning substrate according to claim 8, wherein a photocatalyst-containing vascular cell adhesion layer is formed on the base material,
the photocatalyst-containing vascular cell adhesion layer contains at least: a photocatalyst; and a vascular cell adhesive material which has adhesive properties to a vascular cell, and is to be decomposed or denatured by an action of a photocatalyst upon irradiation with energy, and
in the vascular cell adhesion-inhibiting portion, the vascular cell adhesive material have been decomposed or denatured by an action of a photocatalyst upon irradiation with energy.

11. The vascular cell culture patterning substrate according to claim 7, wherein a photocatalyst-containing layer and a vascular cell adhesion layer are formed on the base material,
the photocatalyst-containing layer contains at least a photocatalyst,
the vascular cell adhesion layer contains a vascular cell adhesive material which has adhesive properties to a vascular cell, and is to be decomposed or denatured by an action of a photocatalyst upon irradiation with energy, and
in the vascular cell adhesion-inhibiting portion, the vascular cell adhesive material have been decomposed or denatured by an action of a photocatalyst upon irradiation with energy.

12. The vascular cell culture patterning substrate according to claim 8, wherein a photocatalyst-containing layer and a vascular cell adhesion layer are formed on the base material,
the photocatalyst-containing layer contains at least a photocatalyst,
the vascular cell adhesion layer contains a vascular cell adhesive material which has adhesive properties to a vascular cell, and is to be decomposed or denatured by an action of a photocatalyst upon irradiation with energy, and
in the vascular cell adhesion-inhibiting portion, the vascular cell adhesive material have been decomposed or denatured by an action of a photocatalyst upon irradiation with energy.

13. The vascular cell culture patterning substrate according to claim 7, wherein a vascular cell adhesion layer is formed on the base material,
the vascular cell adhesion layer contains a vascular cell adhesive material which has adhesive properties to a vascular cell, and is to be decomposed or denatured by an action of a photocatalyst upon irradiation with energy, and
in the vascular cell adhesion-inhibiting portion, the vascular cell adhesive material have been decomposed or denatured by an action of a photocatalyst upon irradiation with energy.

14. The vascular cell culture patterning substrate according to claim 8, wherein a vascular cell adhesion layer is formed on the base material,
the vascular cell adhesion layer contains a vascular cell adhesive material which has adhesive properties to a vascular cell, and is to be decomposed or denatured by an action of a photocatalyst upon irradiation with energy, and
in the vascular cell adhesion-inhibiting portion, the vascular cell adhesive material have been decomposed or denatured by an action of a photocatalyst upon irradiation with energy.

15. A method for manufacturing a blood vessel, wherein the vascular cell is cultured using the vascular cell culture patterning substrate according to claim 7.

16. A method for manufacturing a blood vessel, wherein the vascular cell is cultured using the vascular cell culture patterning substrate according to claim 8.
